(19) United States
(12) Patent Application Publication
(10) Pub. No.: US 2009/0246125 A1
(43) Pub. Date: Oct. 1, 2009

(54) MAGNETIC RESONANCE CONTRAST MEDIA

(76) Inventors: Stephen Lawrence Atkin, Hull (GB); Stephen Thomas Beckett, York (GB); Grahame Mackenzie, Hull (GB)

Correspondence Address: Weaver Austin Villeneuve & Sampson LLP, P.O. BOX 70250, OAKLAND, CA, 94612-0250, US

(21) Appl. No.: 11/721,782

(22) PCT Filed: Dec. 14, 2005
(86) PCT No.: PCT/GB2005/004824

§ 371 Date: Jun. 14, 2007

(30) Foreign Application Priority Data
Dec. 16, 2004 (GB) 0427520.2

(51) Int. Cl. A61K 51/00; C12N 1/19; C12N 5/04; C12N 1/12; A61K 49/00

(52) U.S. Cl. 424/1.17; 435/254.1; 435/410; 435/257.1; 424/9.3; 424/9.4; 424/9.5

(57) ABSTRACT

An image enhancing agent of a form comprising an effective quantity of an image enhancing material chemically or physically bound to or encapsulated within a support comprising, preferentially, an exine coating of spores of various plants or fungi, optionally with further excipients.

This invention relates to image enhancing agent for medical diagnostics in particular for magnetic resonance imaging.

When using magnetic fields or electromagnetic waves as diagnostic techniques to image the human body it is normally necessary for the organ under investigation to contain some material which interacts with the field or wave in order to increase the contrast or obtain better definition.

The two types of image enhancing materials are used for magnetic resonance imaging. These either act predominantly either on the T1 relaxation which results in signal enhancement and positive contrast or on the T2 relaxation which results in signal reduction and negative contrast.

The positive image enhancing materials are typically small molecular weight compounds containing the elements gadolinium, manganese or iron. These have unpaired electron spins in their outer shells and long relaxivities. Positive materials are small particulate aggregates, which produce predominantly spin-spin relaxation effects. Particles smaller than 300 nanometres produce substantial T1 relaxation. Conventional gadolinium complexes cannot be administered orally into the bloodstream due to their difficulty in penetration of the gastrointestinal membrane. These complexes can however be injected into the bloodstream, but are removed after one pass, so their effectiveness is very short lived. In addition these complexes soon become very much dispersed and cannot be focused on a particular organ. Similarly barium meal, used to enhance X-rays, soon becomes dispersed and consequently needs to be used at a high dose. It is also restricted to use in the gut.

By attaching to or encapsulating the appropriate material within the exine coating of a spore, we have developed an image enhancing agent that can be used with magnetic resonance imaging, X-ray, gamma cameras, ultrasound or any other technique affected by this material. These can be used in either the gut or blood stream and their size can be chosen to be optimal for particular parts of the body. As relatively large amounts of material can be retained within a small volume, more intense signals can be obtained from a limited region thereby improving resolution. In addition, it is possible to retain the material in the bloodstream for more than one pass.

Sporopollenins form the exine coatings of spores or pollens of various plants, fungi and algae. Sporopollenins may be separated from spores or pollens by successive treatment with solvents, alkali and acid to remove the cellulose wall and any carbohydrates or proteins. Enzymic methods have also been used. Sporopollenins have chemically and physically stable, carotenoid-like structures.

PCT/GB04/002775 describes a pharmaceutical dosage form in which an active agent is attached physically or chemically to the exine coating of spores of a plant, fungus or algae or fragments thereof. It is however possible to attach or encapsulate non-active materials to these exine coatings or fragments thereof. These materials have no direct influence on the health of the individual or animal containing them. On the other hand, because they are present in a relatively high concentration within or on the surface of the coating they are able to affect magnetic or electromagnetic fields.

According to a first aspect of the present invention there is provided a magnetic resonance image enhancing agent, comprising an effective quantity of an image enhancing material chemically or physically bound to or encapsulated within a support selected from: an exine coating of spores or pollens of a plant, fungus, or algae, or fragments thereof.

In preferred embodiments the support comprises sporopollenin. Sporopollenins or other exine coatings of spores or pollen grains or related microspores have the advantage that they are chemically and mechanically stable, are convenient to use and administer and are easy and cheap to prepare. Sporopollenins may be generally free of leachable impurities. Sporopollenins may be functionalised or the hollow interior may be filled to provide a high image enhancing material loading capability.

The image enhancing material for magnetic resonance imaging is preferably a metal complex, chelate or other derivative, wherein the metal is selected from gadolinium, manganese, iron or mixtures thereof.

Alternatively, but not essentially, the material may be a radionucleotide for use where the imaging technique is a gamma camera or similar device.

In a further embodiment the air, or other gas, inside an otherwise empty exine shell acts as an image enhancing material for ultrasound diagnosis. The exine shell may be coated with another coating material to prevent liquids entering the shell. Non-exclusive examples of these coating materials are waxes, fats and metals.

The exine coating in accordance with this invention has the advantage that it is stable in acid or alkaline media and able to withstand exposure to temperatures up to 250° C. The coating is not readily destroyed within the gut. On the other hand the exine coating is biodegradable in the blood to allow release of image enhancing agent. The degradation products are essentially non-toxic and are unlikely to show an inflammatory response. Residence time in the gastrointestinal tract may be low for a proportion of the exines from spores smaller than 40 micron, which persorb into the blood stream within minutes of passing into the mouth. This proportion will depend upon the size of the exine, the method of delivery and other factors. Degradation may occur within the bloodstream, permitting efficient administration of the image-enhancing agent, for example within a period of several minutes e.g. 10 minutes.

The image enhancing agent may comprise a conjugate, chelate or complex obtained by chemically bonding, preferably covalently bonding the contrast agent to a carrier or substrate comprising a spore, sporopollenins or other spore derivative. Although covalent bonding is preferred for most applications, ionic bonding, hydrogen bonding or van der Vaals bonding may be used, particularly in applications in which strong bonding of the drug to the carrier may not be required. The active image enhancing agent may be reacted directly with the sporopollenin to produce a bioconjugate. However, in preferred embodiments of this invention the sporopollenin or other exine coating is functionalised so that the contrast medium can be attached by a suitably stable covalent or other chemical linkage. For example, but not essentially, the functional group may be a primary amine, polyamino, thiol, carboxylic acid, amino acid, polyhydroxyl, or halogeno group.

For oral delivery the linkage may be selected to be stable in acid solutions so that the medium and support can pass through the stomach into the intestinal tract.

Alternatively the image enhancing material may be physically bonded to the carrier or substrate.

The material when physically bound may be adsorbed on to the support.

Alternatively and more preferably the material is retained within the cavities of the hollow spore coatings. This enables very high loadings of the material to be obtained. More than equal weight for weight has been achieved in certain circumstances.

According to a preferred aspect of the present invention, a method of making an image-enhancing agent comprises the steps of:
optionally contacting an exine coating with a penetration aiding liquid,
contacting the exine coating with a material, which will react with a field or
wave, and allowing the image enhancing material to penetrate into the interior of the coating.
removing any penetration aiding liquid and allowing a coating to dry to retain the material within the coating.

The field or wave may be selected from: magnetic, ultrasound and electromagnetic fields or waves.

A preferred penetration aiding liquid is selected from the group consisting of C₁ to C₄ alcohols, preferably ethanol or aqueous C₁ to C₄ alcohol, preferably aqueous ethanol.

The exine coating may be soaked in a solution of the image enhancing material in the penetration aiding liquid. Alternatively, the exine coating may be soaked in the penetration aiding liquid prior to contacting with the image enhancing material.

Pressure or vacuum may be applied to increase the rate of penetration of the image enhancing material into the exine coating. Use of pressure or vacuum may avoid the need for use of a penetration aiding fluid.

Image enhancing agents in accordance with this invention confer many advantages. The gastric mucosa and small intestine can be outlined. Absorption of the medium allows the blood supply from the stomach and the portal circulation to be outlined. Primary and secondary metastases involving the hepatic and gastrointestinal circulation may be identified. Localised absorption, for example, in the stomach and oesophagus may be used to detect abnormalities such as ulceration, malignancy, pernicious anaemia in the stomach and polyps. The agents can be used advantageously to investigate for Crohn's disease, ulcerative colitis, polyps, causes of intestinal obstruction, malignancies and lymphoma of the small and large intestine. Sources of abnormal bleeding can be highlighted throughout the gut where currently there is no obvious conventional means of doing this. The agents can be used advantageously to investigate the uterus, fallopian tubes, and other fertility and gynaecological problems. The agent may also be used as an enema either introduced through the stomach (small bowel enema) or to investigate the rectum and large intestine via the rectal route.

A particular advantage is that it provides an alternative to use of a barium meal that is more pleasant to ingest as well as providing greater resolution at the same concentration.

Media consisting of or comprising the exine coatings of spores, pollens or other spore or pollen derivatives have distinct advantages on account of their ability to persorb rapidly into the blood following oral administration. In addition they can be readily derivatised to attach a wide range of image enhancing materials, having different imaging profiles, solubilities and stabilities. Such media are chemically and morphologically consistent, are capable of protecting acid labile molecules and are non-toxic. Also, there is no allergic response when the exine coating is taken orally due to all of the proteins associated with such responses having been removed from the raw plant spore or pollen. Decomposition of an exine coating occurs rapidly in blood allowing a quick release of the contrast agent, but may be slower than current agents allowing concentration of the agent in areas of abnormal vasculature, or in abnormalities of the solid organs such as the liver. Exine coatings, when taken orally have been shown to continue entering the blood stream for a period of more than 40 minutes, so continuous monitoring is possible over this period. Exine coating particles may retain their size and morphology during administration and absorption. Uniformity of size and morphology of the exine coating of spores from particular species enables the media to be optimised in accordance with the image enhancing material loading and mode of delivery.

Large exine coatings can be found e.g. from Cuburbita these are 250 microns in size. When they are above 40 microns they are very unlikely to pass from the gut into the blood stream. Because of their large volumes they are able to encapsulate relatively large amounts of the image enhancing material. They are resistant to acid or alkali and so can pass through the gut providing enhanced imaging over a long period of time.

The attachment can be chemical, such that the agent remains with the exine throughout. Alternatively with physical attachment, the method of attachment can be designed to allow release over a period. In a further embodiment coatings of the exines can be formulated to provide a delayed release, for example using an enteric coating.

The exine coating of smaller spores or pollens can be used in several different ways. As a proportion, when taken orally, migrate rapidly into the blood stream, this effect can be used to enhance the imaging in the blood and/or the gut at the same time. Alternatively they can be injected directly into the blood stream. The exine coating will be destroyed in the bloodstream during a period of minutes, but the agent will provide an enhanced image over longer periods, because new loaded exine coatings will continue to enter the system and will concentrate in areas of interest and abnormality. In this situation much lower dosages will be required than may be conventionally used due to the property of focussing and pooling that will occur at areas of abnormality. Currently available gadolinium complexes are mainly removed from the system after a single pass within seconds or minutes.

The source of spore used can be chosen to produce the appropriate size for the diagnostic investigation. For example the spores may be large enough to lodge in vessels, obstructions or partial blockages, thereby providing a particularly intense image at the point of interest. Differently sized spores may be chosen to remain in the vasculature and then to the enhancing material to go into the cell when the exine is destroyed. High loading of the exine coating with diagnostic material at low quantity of exine for injection can give different information in comparison to low loading of the exine with diagnostic material at higher quantity of exine for injection.

The source of the spore can also be chosen so as to target specific regions of the lung, when the exine is breathed in. For example, Aspergillus niger gives 4 micron exine shells, which will penetrate deeply into the lung, whereas Lycopodium, with 25 micron exine shells can be used to investigate nasal and upper airways.

Air microbubbles are known to affect ultrasound signals and indeed hollow sugar-based particles are sold as image enhancement agents. The use of empty exine coatings have the advantage that they can be used in the gut because they are not affected by acid or alkali. Furthermore, if coated, air-filled coatings can be tracked the whole way down the gut. Moreover their size can be chosen according to the particular application.

Image enhancing agents of the present invention may be provided for human or veterinary use. The diagnostic method may be magnetic resonance imaging, x-ray or ultrasound or any other technique where the imaging signal is enhanced by the material within or attached to the exine coating.

The invention is further described by means of example but not in any limitative sense.

Sporopollenin may be isolated by harsh treatment of spores or pollens with a combination of organic solvents and strong acids and alkalis.

### EXAMPLE 1

### Isolation of Sporopollenin from Lycopodium Clavatum

Lycopodium (250 g, commercially available from Fluka) was suspended in acetone (700 ml) and stirred under reflux for 4 h. The solid residue was filtered, washed with fresh acetone, transferred back to the reaction flask and resuspended in potassium hydroxide solution (850 ml, 6% w/v in water). The mixture was then stirred under refluxed for 6 h. The residue was filtered, washed copiously with hot water, transferred back to the reaction flask, and the hydroxide treatment was repeated. After filtration the solid material was washed with hot water, hot ethanol, and water again. The residue was stirred under reflux in ethanol (750 ml) for 2h, filtered and washed sequentially with fresh ethanol and dichloromethane. The resulting solid was resuspended in fresh dichloromethane (750 ml), stirred under reflux for 2 h, removed by filtration and dried in air for 24 h.

The filtered particles were then suspended in orthophosphoric acid (85%, 800 ml), stirred under reflux for 5 days and filtered. The residue was washed with copious amounts of hot water and sucked dry. The orthophosphoric acid treatment and drying was repeated. The particles were then washed with hot, water, ethanol, and dichloromethane. Finally the solid was stirred under reflux in ethanol (800 ml) for 2 h, filtered and washed with dichloromethane to yield sporopollenin (50 g) that was air-dried and then vacuum dried.

### EXAMPLE 2

### Loading the Exine with an Image Enhancing Material

### Copper(II)EDTA Complex

A stirred mixture of EDTA, as the disodium salt (6 g) and copper chloride dihydrate (2.5 g) in water (25 ml) was heated for 1.5 h. The cooled solution was filtered and the resulting saturated solution of Cu(II)EDTA complex was stirred with ethanol (2 ml) and sporopollenin exine (0.5 g, not compressed) for 2 h, washed well with water to remove any copper complex from the surface and then dried at 110° C. to constant weight. The loading of Cu(II)EDTA was found to be 2.69 mmol/g based on mass gain. The exine particles showed no complex on their exterior by SEM.

### EXAMPLE 3

### The Slow Release of the Image Enhancing Material Within an Aqueous Medium

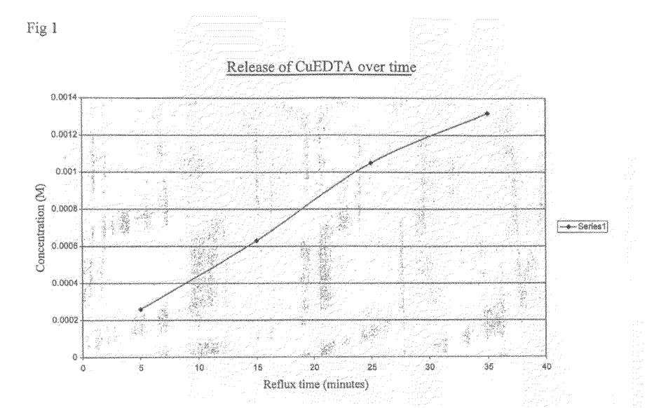

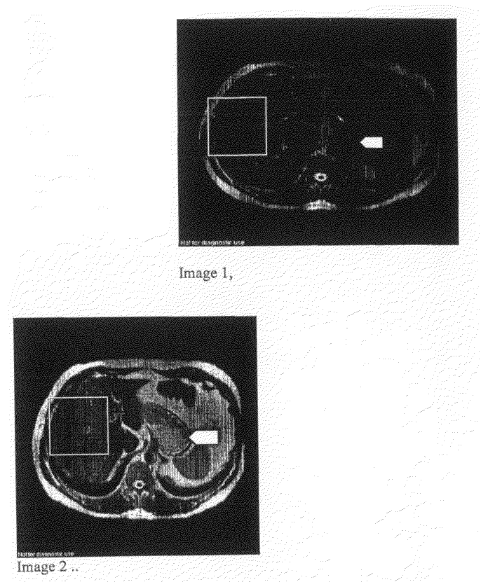

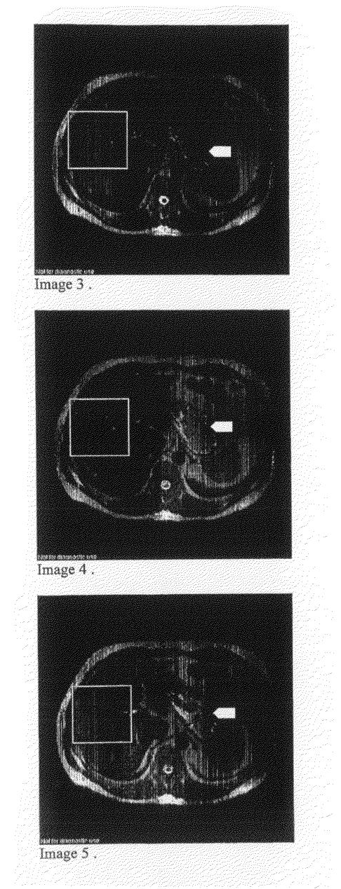

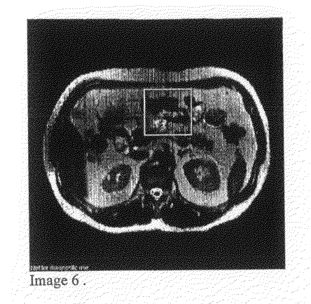

The particles, as prepared in Example 2, were refluxed in water (50 ml) over 50 minutes and the evolution of copper from the exines was determined by FAAS (Furnace Atomic Absorption Spectrometry). FIG. 1 shows the relationship obtained between the concentration of Cu(II)EDTA released over time. It can be noted that even after 30 minutes of refluxing not all of the encapsulated material had been released. The graph shows an original loading of, at least, 2.64 mmol/g.

### EXAMPLE 4

### The Use of Coatings to Reduce Release of the Image Enhancing Material in an Aqueous Medium

Particles were prepared as in Example 2. They were then coated in gum Arabic such that the surface was covered. The exine coatings remained as individual particles. The procedure set out in Example 3 was then repeated. The loaded exines were allowed to stand in a 1% aqueous starch solution for 25 minutes and then filtered and dried. The coated particles were refluxed for 30 minutes and dried. The starch coating was found to reduce the loss of Cu(II)EDTA by more than 50%.

### EXAMPLE 5

### The Use of a Chemical Reaction Within the Exine Coating to Reduce Release of the Image Enhancing Material in an Aqueous Medium

Solution A was prepared by mixing 8.5 g of silver nitrate with 22.5 ml of water and 2.5 ml of ethanol. Solution B consisted of 3 ml of concentrated hydrochloric acid in 17 ml of water.

0.5 g of the exine coating from Lycopodium clavatum was compressed under a pressure of 10 tonnes to form a tablet. This tablet was added to 15 ml of solution A and the mixture was stirred for 2 hours. The mixture was filtered and washed quickly with 15 ml of distilled water before the particles were added to solution B and stirred for 2 hours. The exines were then filtered and dried. Electron microscopy coupled with X-ray showed that the exines contained silver chloride crystals.

### EXAMPLE 6

### The Preferential Attachment to an Alginate (Gaviscon-Registered Trade Mark)

In order to simulate what might happen in the gut 2 g of sporopollenin was prepared and loaded with a copper complex as for Examples 1 and 2. It was then mixed with 20 ml of Gaviscon (registered trade mark) (an alginate formulation used to alleviate heartburn) and added to 100 ml of water and acid at a pH of 1. The mixture was shaken vigorously before leaving it to settle. The acidified water remained clear and the loaded sporopollenin particles were seen within the Gaviscon, which was floating on top. It was therefore expected that the image enhancing agent could be used to trace the position of Gaviscon in the gut.

### EXAMPLE 7

### MRI Imaging Around the Gut Wall Using Gaviscon

The procedures used in Examples 1 and aqueous alcohol and vacuum were used to load sporopollenin with Gadolite 60 (sucrose ester). This was then mixed with a single dose of Gaviscon. MRI was then used to scan the stomach. A very bright image was seen of the Gaviscon against the gut wall.

### EXAMPLE 8

### The Use of Hollow Exine Coatings to Aid Ultrasound Detection

Exine coatings of Lycopodium were prepared as in Example 1. 100 mg of particles were coated with starch (using a 1% aqueous solution as in Example 4) and a further 100 mg were filled with cocoa butter using vacuum and ethanol as a penetration aiding liquid. Both were suspended in 10 ml of water and put in a syringe with a 1 mm bore needle. Both suspensions were injected into a 8 cm×12 cm×3 cm piece of bone-free meat. The meat was examined using a Siemens Autares machine with a 10 MHz linear probe. The ultrasound detected the coated spores 1.3 cm into the meat as a clean shadow with a comparable reflection to calcium. The probe was unable to detect the fat filled exine coatings.

### EXAMPLE 9

### In vitro Studies Using Encapsulated Gd Complex

### Sample Preparations

A solution of Gd complex was added to Sporopollenin (AHS 25μ and SSSP3 40μ). The mixture was stirred to afford a homogenous mixture and was left under vacuum over P₂O₅ for 2 h. The solutions of Gd were prepared in water/EtOH (4:1) as shown below:
Solution A: 0.1 mL of Gd complex in 100 mL of solution.
Solution B: 0.2 mL of Gd complex in 100 mL of solution.
Solution C: 1 mL of Gd complex in 100 of solution.
Solution D: 2 mL of Gd complex in 100 of solution.

| SAMPLE | | | [Gd] | |
|---|---|---|---|---|
| ID | Sporopollenin/mg | Gd solution/mL | V/V | Gd g/Sp g |
| AT022 | 107.2 (AHS 25μ) | 0.4 Solution A | 0.001 | 1.75 10⁻³ |
| AT023 | 116.9 (AHS 25μ) | 0.4 Solution B | 0.002 | 3.21 10⁻³ |
| AT024 | 110.2 (AHS 25μ) | 0.4 Solution C | 0.01 | 17.02 10⁻³ |
| AT025 | 110.0 (AHS 25μ) | 0.4 Solution D | 0.02 | 34.11 10⁻³ |
| AT026 | 104.3 (SSSP3 40μ) | 0.4 Solution A | 0.001 | 1.80 10⁻³ |
| AT027 | 108.1 (SSSP3 40μ) | 0.4 Solution B | 0.002 | 3.47 10⁻³ |
| AT028 | 107.4 (SSSP3 40μ) | 0.4 Solution C | 0.01 | 17.47 10⁻³ |
| AT029 | 103.2 (SSSP3 40μ) | 0.4 Solution D | 0.02 | 36.36 10⁻³ |
| AT033 | 99.2 (AHS 25μ) | 0.2 Solution A | 0.001 | 0.94 10⁻³ |
| AT034 | 106.5 (AHS 25μ) | 0.2 Solution B | 0.002 | 1.76 10⁻³ |
| AT035 | 109.3 (AHS 25μ) | 0.2 Solution C | 0.01 | 8.58 10⁻³ |
| AT036 | 98.0 (AHS 25μ) | 0.2 Solution D | 0.02 | 19.14 10⁻³ |
| AT037 | 104.0 (SSSP3 40μ) | 0.2 Solution A | 0.001 | 0.90 10⁻³ |
| AT038 | 109.2 (SSSP3 40μ) | 0.2 Solution B | 0.002 | 1.71 10⁻³ |
| AT039 | 107.1 (SSSP3 40μ) | 0.2 Solution C | 0.01 | 8.76 10⁻³ |
| AT040 | 106.2 (SSSP3 40μ) | 0.2 Solution D | 0.02 | 17.66 10⁻³ |

### EXAMPLE 10

### Encapsulation of Magnevist (Gd) in Sporollenin. MRI

A solution of Gd complex (30 ml) was poured into Sporopollenin (5 g of CFS 25μ and SSSP4/5 40μ) Both samples were prepared in 50 ml centrifuge tubes, and the mixture was left to afford a wet sample, both sediment and a supernatant liquid. The solutions of Gd were prepared in water/EtOH (4:1) as shown below:
Solution: 0.06 mL of Gd complex in 1000 ml of solution.

After 12 days the supernatant liquid was removed (3 ml in each sample) and the samples were tested by MRI.

| SAMPLE ID | Sporopollenin/g | Gd solution/mL | Gd g/Sp g w/w |
|---|---|---|---|
| AT063 | 5.0 (CFS 25μ) | 27 | 0.15 10⁻³ |
| AT064 | 5.0 (SSSP4/5 40μ) | 27 | 0.15 10⁻³ |

Using a 3T MRI scanner all preparations in 25 mm particles showed signal intensity with perhaps the best being 0.15×10⁻³ Gd g/Sp g;. This concentration of aqueous Magnovist was then loaded into 25 and 40 mm particles (5 g). The 40 mm particles showed a significantly stronger image consistent with the greater quantity loaded within each exine.

This example shows that exines alone cannot be seen by MRI technology, but encapsulated gadolinium complexes within the exines can be readily visualised.

### EXAMPLE 11

### In vitro Studies with Encapsulated Fats

The following free liquid oils we investigated in vitro: cod liver oil, sunflower oil, soybean oil, echium oil and rapeseed oil. All appeared at a similar intensity when scanned.

A sample of 6.6 g of cod liver oil encapsulated into 3.3 g of sporopollenin was investigated in vitro in the magnet. Modest intensity was observed by comparison to unloaded free liquid oil. Sporopollenin exines alone showed nearly no image.

Exines loaded with oil were not affected by simulated gastric acid pH 2 with pepsin after 1 hour of incubation.

This example shows that exines alone cannot be seen by MRI technology, but encapsulated oils within the exines can be readily visualised and used as MRI contrast agents.

### EXAMPLE 12

### In vivo Studies Investigating the Effect of Fat Filled Sporopollenin on MRI Images

A blank was prepared of the empty 40 mm exines. This was taken with 150 ml full fat milk. Both the 25 & 40 mm exines (15 g in each case) were loaded with fish oil at the levels of sporopollenin-oil 1:3 w/w and 1:5 w/w. These were taken with full fat milk.

### EXAMPLE 13

### Studies with Human Volunteers

MR imaging of the stomach and liver was performed using a 3T MRI machine giving excellent definition of the structures, using a programme specifically to enhance fat.

It was noted that the preparation looked dry and did not smell or taste of fish oil.

6.67 g of fish oil Fish encapsulated in 3.3 g of lycopodium clavatum sporopollenin exine was given orally and washed down with 150 ml of full fat milk.

MR imaging was performed every five minutes for 20 minutes.

After 1 hour further imaging was performed and 200 ml water was given

Blood was withdrawn every 10 minutes

The results are show in Images 1 to 6.

Following the sporopollenin ingestion, bright imaging of the stomach detailing the stomach mucosa was seen. (Image 1)

Before the ingestion of oil filled exine. Quiescent stomach (arrow) and region of interest in liver (highlighted by box) are visible.

Increasing intensity of imaging of the stomach occurred at 10 minutes (Image 2) with increased signal in the smaller blood vessels of the liver (Image 3 and 4). There was increased intensity of the signal up to 20 minutes with subsequent reduction of the signal at 30 minutes (image 6) This indicated transport of the oil filled sporopollenin exines through the portal circulation to the liver. During this period no signal was seen in the duodenum suggesting that direct absorption through the stomach had occurred.

At 1 hour fine detail of the mucosa of the duodenum was observed (Image 6).

At one hour ingestion of water caused complete loss of the residual signal in the stomach

Blood withdrawn indicated oil filled exines up to 30 minutes in the circulation.

There was no absorption of exines of 40 μm

Implications of the in vivo Studies of Encapsulated Oils

Detailing of the stomach mucosal surface and architecture:
1 This may be particularly important as there are no current agents that may image the architecture of the stomach that may have utility in imaging ulcers, tumours, polyps and other stomach pathology using MRI.
2 This also indicate that lipid filled exines have a role as a specific imaging agent in their own right for the purpose of detailing the stomach/mucosa surface architecture.
3 The use of gadolinium complexes within exines are likely to give greater visualisation.

### Signal in the Liver:

1 This indicates that the exine was transported in the portal circulation and can be used to diagnose disorders of this system.
2 This indicates that the exine encapsulating oil or other contrast agents have an important use as an agent to image the liver and have specific utility for the diagnosis of tumours, cirrhosis, inflammation, cysts and other liver pathology.

### Signal in the Duodenum:

1 This may be particularly important as there are no current agents that may image the architecture of the duodenum that may have utility in imaging ulcers, tumours and other intestinal pathology using MRI.
2 This also indicate that lipid filled exines have a role as a specific imaging agent in their own right for the purpose of detailing the stomach/mucosal surface architecture.
3 The use of gadolinium complexes within exines are likely to give greater visualisation.

### Blood Results

Exines isolated from the blood stream were subject to confocal microscopy. The oil within the exine was visualised showing that the exine had transported the exine through the gastrointestinal mucosa and into the circulation (Image 5). This oil was then progressively released in the circulation (Image 6).

### Interpretation of the Blood Results

1 These results showed that a contrast agent and in this case oil was transported into the blood stream and progressively released, thus indicating that other contrast agents such as gadolinium, iron and manganese can also be transported.
2 That 25 mm exines were isolated up to 30 minutes indicated that they had been transported by the portal circulation and had passed through the liver and into the general circulation. This means that abnormal pathology past the liver in distant organs such as primary, secondary and metastatic tumour vessels, sites of bleeding will also be visualised by this exine technology encapsulating gadolinium, iron or manganese.
3 That the 40 mm exines were not absorbed indicated that they have utility in the visualisation of the gastrointestinal system for the identification of primary, secondary tumours, polyps, inflammation (such as Crohn's disease, ulcerative colitis), external compression (such as tumours), mucosal abnormalities incorporating the proximal, mid and distal small intestine, the large intestine and rectum, including intestinal obstruction
4 The agents can be used advantageously to investigate the uterus, fallopian tubes, and other fertility and gynaecological problems by MRI.
5 These agents may direct surgical retrieval of nodes identified with Gd-DTPA.

1. An image enhancing agent for medical diagnostics comprising an effective quantity of an enhancing material chemically or physically bound to or encapsulated within a support selected from: an exine coating of spores or pollens of a plant, fungus or algae or fragments thereof.

2. (canceled)

3. (canceled)

4. An agent as claimed in claim 1 selected from: tablets, capsules, chewy sweets, ovules, elixirs, solutions and suspensions.

5. An agent as claimed in claim 1 wherein the contrast material is a metal complex, chelate or other derivative.

6. An agent as claimed in claim 1 wherein the contrast material is a radio-nucleotide.

7. An agent as claimed in claim 1 wherein the contrast material is a gas inside the exine coating.

8. An agent as claimed in claim 3, wherein the metal is gadolinium, manganese or iron or a mixture thereof.

9. A diagnostic method comprising:
administering the image enhancing agent as claimed in claim 1 to an individual; and
subjecting the individual to magnetic resonance imaging.

10. A diagnostic method comprising:
administering the image enhancing agent as claimed in claim 1 to an individual; and
subjecting the individual to an X-ray.

11. A diagnostic method comprising:
administering the image enhancing agent as claimed in claim 1 to an individual; and
subjecting the individual to an ultrasound.

12. A diagnostic method comprising:
administering the image enhancing agent as claimed in claim 1 to an individual; and
subjecting the individual to gamma camera imaging.

13-15. (canceled)
